(19) United States
(12) Patent Application Publication
(10) Pub. No.: US 2008/0273658 A1
(43) Pub. Date: Nov. 6, 2008

(54) Machine for accurately calibrating a mammographic X-ray machine and process for operating, calibrating and analyzing same

(76) Inventor: Donald Gary Pellinen, Levermore, CA (US)

Correspondence Address: Donald G. Pellinen, 1208 Asti Ct., Livermore, CA, 94550, US

(21) Appl. No.: 12/150,586

(22) Filed: Apr. 28, 2008

Related U.S. Application Data
(60) Provisional application No. 60/927,783, filed on May 5, 2007.

(51) Int. Cl. A61B 6/04

(52) U.S. Cl. 378/37

(57) ABSTRACT

A machine and procedures for accurately calibrating a mammographic X-ray machine to give accurate, absolute readings of breast tissue density. A sensor to automatically measure the distance apart of the breast retaining plates to a high accuracy, an accurately calibrated sensor to measure the voltage applied to the X-ray tube, an accurately calibrated sensor to measure the current flow to the X-ray tube, A computer interfaced scanning analogue to digital converter to accurately read and store the readings of the spacing sensor and the voltage and current sensors, A digital computer to control and read the scanning analogue to digtal converter, read the radiation detector array used to record the image, to insert the calibration plates to do the calibrations and analyze the data and a program to compute the radiation for optimizing the radiation exposure for a given patient.

### CROSS REFERENCE TO RELATED APPLICATIONS

This application is based on provisional application Ser. No. 60/927783, filed on May 5, 2007.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of medical diagnostics and more specifically to a machine for accurately calibrating a mammographic X-ray machine and process for operating, calibrating and analyzing same.

Mammography is used as a diagnostic tool to locate breast cancers. Breast cancer is second only to lung cancer as a cause of death of American women.

Estimated new cases and deaths from breast cancer in the United States in 2008 are:

New cases: 182,460 (female); 1,990 (male)

Deaths: 40,480 (female); 450 (male)

The first reference to mammography was a study of three thousand cases by a Berlin physician, A. Salomon in 1913. There was little practical use of mammography until a study from 1960 was made.

Beginning in 1960 a major ten-year study was conducted where half the women tested were given a physical exam and half were given a physical exam plus a mammogram. In women over fifty ears of age there were one third fewer deaths among the women having the mammograms as well as the physical examination. This resulted in a recommendation that women considered in danger be given a mammogram every on or two years.

During this period the CGR Senographe (GE Medical Systems, Milwaukee, Wis.), introduced in 1965. This was the first dedicated mammography unit, and its molybdenum anode and filter had many of the essential elements found in modern equipment. Systems from other manufacturers followed.

U.S. Patents

U.S. Pat. No. 5,371,777—Automatic x-ray exposure unit for mammography

Describes a system where the exposure for film is calculated from the thickness exposed and setting the time of the exposure.

U.S. Pat. No. 5,406,612—Method of and apparatus for standardizing and monitoring image quality in mammography

Provides a method for standardizing a radiographic film exposure. Uses light rather than X-ray for some of the calibration. Labor intensive.

Pat. No. 5,544,238—Method of and apparatus for standardizing and monitoring beam quality in mammography

Describes method of standardizing an X-ray beam for film exposure. System is largely manual and very labor intensive and time consuming.

U.S. Pat. No. 6,574,499—Mammography method and apparatus

Describes a multisensor system using ultrasonic and X-ray systems.

U.S. Pat. No. 6,583,420—Workstation interface for use in digital mammography and associated methods

Very general on describing a number of enhanced viewing techniques and attachments. Supposedly will work with almost any ray or particle.

U.S. Pat. No. 6,630,937—Workstation interface for use in digital mammography and associated methods

Describes a computer interface for analyzing data on radiographs.

U.S. Pat. No. 6,748,044—Computer assisted analysis of tomographic mammography data

Describes the use of computer to analyze data from mamographies.

U.S. Pat. No. 6,816,569—X-ray diagnostics installation for mammography examinations

Primarily describes the physical installation.

U.S. Pat. No. 6,956,975—Method for improving breast cancer diagnosis using mountain-view and contrast-enhancement presentation of mammography

Describes method of enhancing image from radiography. Does not describe anything about accurately calibrating the image.

U.S. Pat. No. 6,999,554—X-ray diagnostic apparatus for mammography examinations

Describes the arrangement of the mechanical components of the X-ray system.

U.S. Pat. No. 7,120,224—X-ray imaging apparatus and method for mammography and computed tomography

Is primarily a system to improve radiographic images by developing a monoenergetic Z-ray source. Does not describe calibrations.

U.S. Pat. No. 7,123,684—Full field mammography with tissue exposure control, tomosynthesis, and dynamic field of view processing

Describes a system for semiautomated setup and control for a mammographic X-ray system

U.S. Pat. No. 7,147,372—Device and system for improved imaging in nuclear medicine and mammography

The abstract describes a broad range of improvements for virtually every type of radiography including calibrations. The does not describe the calibrations.

U.S. Pat. No. 6,654,445 Device and method for determining proportions of body materials by inventors: John A. Shepherd, Steven R. Cummings, Karla Kerlikowske. The patent that comes closest to providing a good calibration is this one. It does not address the variation across a detector array.

Foreign Patents

Pat. No. FR2851359—Radiographic image acquiring apparatus calibrating process for e.g. mammography, involves correcting each image of object e.g. bone, by subtracting current image variation of gray level in relation to primary image of object

Describes a method for subtracting the “gray level” from an image to provide clearer view of different features.

Pat. No. GR2004100155—HUMAN CHEST PHANTOM SETUP FOR THE EVALUATION OF THE IMAGE QUALITY OF RADIOLOGICAL EQUIPMENT

Describes a phantom for checking the radiographic exposure quality of a chest X-ray. Does not quantify the exposure.

Pat. No. US2005276379—Portable, digital X-ray apparatus for producing, storing, and displaying electronic radioscopic images

Describes a portable X-ray system using a pulsed X-ray source and digital readout. Does not describe calibrations.

Publications

Compositional Breast Density as a Risk Factor, http://www.cbcrp.org/research/PageGrant.asp?grant_id=2432

Dissecting a Hidden Breast Cancer Risk, http://www.cpmc.org/professionals/research/programs/sciencearticle.pdf

ESTAR>stopping-powe and range tables for electrons, http://physics.nist.gov/PhysRefData/Star/Text/ESTAR.html

X-Ray Data Booklet, http://xdb.lbl.gov/

X_RAY OPTICS TOOLS, http://www-cxro.lbl.gov/index.php?content=/tools.html

XrayMassCoef/cover.html, Tables of X-Ray Mass Attenuation Coefficients http://physics.nist.gov/PhysRefData/XrayMassCoef/cover.html. This is the NIST database of X-ray absorption coefficients. This has attenuation coefficients for all atomic numbers and many tissues and tissue equivalent materials. Probably the most useful source of medical radiography calculational materials.

Miglioetti et al. “Radiologist Characteristics Associated With Interpretive Performance of Diagnostic Mammography”, JNCI Journal of the National Cancer Institute Advance Access published Dec. 11, 2007. This examined the results of the analysis of more than 35 thousand mammograms and concluded 21% were misdiagnosed. There are a number of earlier reports that give the numbers as higher than 21%.

Currently mammographic radiographs are diagnosed subjectively with much of the information being given by appearance. There is almost no data available on calibrations on the most radiographic systems. Most scientific and technological work and the advances made require quantitative data and standards that are accurately calibrated. Calibrated and accurate data can be easily analyzed and compared using the mathematical and statistical techniques common in most sciences. Long term comparisons can be made of mammograms from different times and changes plotted. The difference between an accurate measurement is if one quoted a distance measurement as 1.2567±0.0012 inches as opposed to a statements like “it is a little more than an inch long”. There is currently an error rate in the diagnosis of over 20% using conventional techniques. There is currently no means of determining the sensitivity of various parts of the sensor against other part and what the picture brightness means in absolute terms.

### BRIEF SUMMARY OF THE INVENTION

The goal of this invention is to provide an accurate tool so breast cancers may be detected and treated more reliably and earlier so treatment may begin before the cancer spreads. If treated at this stage, the mortality rate is zero as opposed to about eighty six percent after it spreads. The saving of lives could be many thousands annually in the US alone.

The primary object of the invention is that it provides an automated means of accurately calibrating a mammographic X-ray machine against absolute, NIST traceable standards.

Another object of the invention is allows use of statistical scientific techniques to analyze data as well as the visual methods now used.

Another object of the invention is increases the sensitivity of the measurement so small cancers may be detected earlier.

A further object of the invention is the calibrator allows a series of radiographs taken over a period of time to be analyzed numerically for small changes in tissue density to detect cancers earlier.

Yet another object of the invention is installed equipment allows the exposure conditions to be automatically calculated and documented with minimal operator intervention.

Still yet another object of the invention is accurately calculating exposure parameters to minimize radiation exposure from over exposure and repeat exposures.

Another object of the invention is Increasing accuracy allows for greater effective sensitivity and lower radiation levels for radiographs.

Another object of the invention is the system will allow for automatically taking and archiving both exposure and calibration data from machine.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a machine for accurately calibrating a mammographic X-ray machine comprising: A standard mammographic X-ray system, An automatic detector array to readout the X-rays transmitted through the tissue giving intensity and spatial distribution, A sensor to automatically measure the distance apart of the breast retaining plates to a high accuracy, An accurately calibrated sensor to measure the voltage applied to the X-ray tube, An accurately calibrated sensor to measure the current flow to the X-ray tube, A computer interfaced scanning analogue to digital converter to accurately read and store the readings of the spacing sensor and the voltage and current sensors, A digital computer to control and read the scanning analogue to digital converter, to read the radiation detector array, to insert the calibration plates to do the calibrations and analyze the data, A module to insert one or more calibration plates in place of the breast to do the calibration exposures, Calibration plates to insert in place of the breast to calibrate the radiographic system, A computer program to read out the detector array or to access the readout system on the X-ray system, A computer program to execute the calibration of the system, A computer program to analyze the data from each point on the detector array and correct the data to give a highly accurate readings over the whole detector, A computer program to analyze the corrected data for easier analysis of the radiograph, A computer program (radiation transport code) to analyze the data from the sensors and compute the radiation for correction and analysis of the data, and A computer program (radiation transport code) to compute the radiation for optimizing the radiation exposure for a given patient.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a cross sectional view of the Calibration system installed on a mammographic X-ray machine.

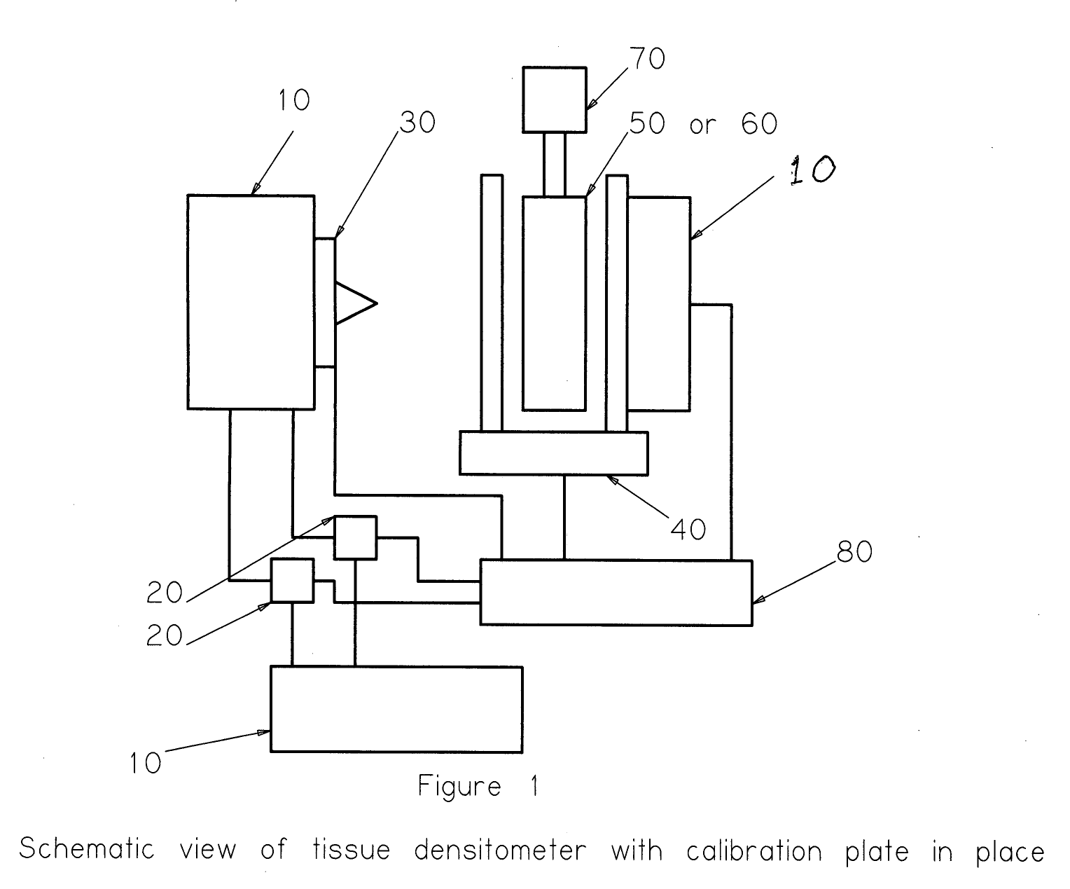

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

10—Mammographic X-ray machine

20—X-ray tube voltage and current sensors

30—X-ray tube radiation output sensor

40—plate spacing sensor

50—Tissue simulator plate for detector normalization

60—Detector calibration plate

70—Insertion mechanism for calibrators

80—Radiation detector plate, either supplied with X-ray machine or added on.

90—Software to drive calibration system and calibrate sensors

100—Display software to generate displays

### Description

Part 10—Mammographic X-ray machine is a commercial medical X-ray machine used for mammography. It consists of a power control unit that supplies voltage and current to the X-ray tube to generate the X-rays. It has a frame and plates to confine the breast for the mammography and a sensor to read out the radiography. Initially the sensor was photographic film that was exposed. Now solid state sensors provide the output in a few seconds without requiring chemical developing of the film and the labor associated with it. If the power control unit does not have them, part 20—X-ray tube voltage and current sensors will be added. Part 30—X-ray tube radiation output sensor will be placed in front of the X-ray tube. Part 40—plate spacing sensor will be added to the plates to measure the spacing and effectively the thickness of the compressed breast being measured. This will be interfaced to and read out by an analogue to digital converter. After the mammograms are made the patient and operator would leave the area and the calibrations made. An exposure would be made with the same settings as the actual exposure without any plates to get the reference level. After that, part 50—detector calibration plate one will be inserted and a radiograph taken. After that 60—second detector calibration plate will be inserted and a radiograph taken. Then both plates would be inserted and the measurement repeated. The insertion and removal of the plates will be done automatically by 70—Insertion mechanism for calibrators. 80—Radiation detector plate, either supplied with X-ray machine or added on. 90 computer-interfaced readouts and actuators will monitor all the parts described. Part 90—Software to drive calibration system and calibrate sensors is a set of computer programs to actuate the system parts and record the data. Part 100—Display software to generate displays.

### Operation

The Automated System to Measure Breast Tissue Density During a Mammogram may be either integrated into a machine being produced or and add on kit for existing machines. A conventional medical radiographic system consists of the controls to drive and power an X-ray tube, which shines X-rays through the subject positioned by some fixture. The X-rays are attenuated by the material in the body that depends on the thickness of the material, the composition of the material and the density of the material. At the X-ray energies normally used for radiography on persons, the attenuation of the X-rays is primarily by the “photoelectric effect” where an X-ray photon interacts with an electron in an atom and gives all it's energy to the atom. The X-ray photon is removed from the beam decreasing the beam intensity. The X-rays that remain will pass into the detector where some will interact with the film or detector and expose the film or give a reading on the sensor. The exposure to a first order is proportional to the number of X-rays per unit area incident on the detector. There is also an interaction called the Compton effect where the interacts with the electron in an atom and gives part of it's energy to the electrons and the photon is redirected at a lower energy and the electron is ejected from the atom.

The Automated System to Measure Breast Tissue Density During a Mammography provides the equipment and techniques to automatically calibrate the system and provide a quantitative measure of breast tissue density. They system operates by allowing the radiologist to set up and take the radiograph as he normally does. The difference here is that the voltage and current and the radiation at the source are accurately measured. The patient is removed from the machine and a tissue simulation calibration plate(s) replaces the patient. An exposure is made at the same settings and radiation level as the exposure and those readings taken. The reading on the detector plate is taken and stored. The insertion mechanism places the detector calibration plates into x-ray beam path to attenuate the beam in a calibrated manner. This is exposed to the same radiation as before and the voltage, current and radiation level measured at the same level as the actual exposure. The radiation detector plate is read out. Readings on areas of the detector plate that are not shielded by tissue or calibration plates may be used to measure the incident radiation.

The voltage, current and radiation data from the exposures are compared. If all three were within acceptable levels, the readings on the exposure to the tissue simulator plate would be analyzed. Readings can vary because of fall off toward the edge or spatial variations across the detector plate or cells being nonfunctional. Since the whole detector was exposed by radiation passing through the same amount of material. A correction factor would be generated for each area of the detector. For example, if the measured exposure at the edge was 2% lower than that in the center, then the readings at the outside would be multiplied by 1.02 to give the correct reading.

To use the calibration block we note that the attenuation of X-rays is given by l=lo*e(−u*d*x) where l and lo are the incident and transmitted X-ray intensity and u is the mass absorption coefficient for the material, d is the density of the material and x the distance transmitted through the material.

A calibration curve is generated by running the normalization curve with the tissue simulator plate(s) and putting in the thickness of the plate(s) to get a calibration curve for that amount of material. The readings on the detector calibration plate are made, the levels computed for the different absorber thicknesses using l=lo*e(−ux). The data can be given a functional dependence using a least-squares curve fit. Using the equations generated and the gap measured the equation log(l/lo)/x=−u*d.

There are a number of ways the calibration plates could be used. If for example, plates of 1, 2, and 4 cm thick were used 8 calibrations points could be made with 0, 1, 2, 3, 4, 5, 6, and 7-cm thick points. A pixel by pixel curve fit done by the computer for each pixel on the readout and the data from the mammograms displayed in terms of tissue density in grams/cm cubed. A goal would be to have the entire detector array calibrated to 0.1% accuracy or better.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A machine for accurately calibrating a mammographic X-ray machine comprising:
A standard mammographic X-ray system;
An automatic detector array to readout the X-rays transmitted through the tissue giving intensity and spatial distribution;
A sensor to automatically measure the distance apart of the breast retaining plates to a high accuracy;
An accurately calibrated sensor to measure the voltage applied to the X-ray tube;
An accurately calibrated sensor to measure the current flow to the X-ray tube;
A computer interfaced scanning analogue to digital converter to accurately read and store the readings of the spacing sensor and the voltage and current sensors;
A digital computer to control and read the scanning analogue to digital converter, to read the radiation detector array, to insert the calibration plates to do the calibrations and analyze the data;
A module to insert one or more calibration plates in place of the breast to do the calibration exposures;
Calibration plates to insert in place of the breast to calibrate the radiographic system;
A computer program to read out the detector array or to access the readout system on the X-ray system;
A computer program to execute the calibration of the system;
A computer program to analyze the data from each point on the detector array and correct the data to give a highly accurate readings over the whole detector;
A computer program to analyze the corrected data for easier analysis of the radiograph;
A computer program (radiation transport code) to analyze the data from the sensors and compute the radiation for correction and analysis of the data; and
A computer program (radiation transport code) to compute the radiation for optimizing the radiation exposure for a given patient.

2. A process for accurately calibrating a mammographic X-ray machine comprising the steps of:
A standard mammographic X-ray system;
An automatic detector array to readout the X-rays transmitted through the tissue giving intensity and spatial distribution;
A sensor to automatically measure the distance apart of the breast retaining plates to a high accuracy;
An accurately calibrated sensor to measure the voltage applied to the X-ray tube;
An accurately calibrated sensor to measure the current flow to the X-ray tube;
A computer interfaced scanning analogue to digital converter to accurately read and store the readings of the spacing sensor and the voltage and current sensors;
A digital computer to control and read the scanning analogue to digital converter, to read the radiation detector array, to insert the calibration plates to do the calibrations and analyze the data;
A module to insert one or more calibration plates in place of the breast to do the calibration exposures;
Calibration plates to insert in place of the breast to calibrate the radiographic system;
A computer program to read out the detector array or to access the readout system on the X-ray system;
A computer program to execute the calibration of the system;
A computer program to analyze the data from each point on the detector array and correct the data to give a highly accurate readings over the whole detector;
A computer program to analyze the corrected data for easier analysis of the radiograph;
A computer program (radiation transport code) to analyze the data from the sensors and compute the radiation for correction and analysis of the data; and
A computer program (radiation transport code) to compute the radiation for optimizing the radiation exposure for a given patient.
